(19) United States
(12) Patent Application Publication
(10) Pub. No.: US 2021/0148918 A1
(43) Pub. Date: May 20, 2021

(54) MODIFYING SURFACE OF A LIVE CELL AND THE USES THEREOF

(71) Applicant: Purdue Research Foundation, West Lafayette, IN (US)

(72) Inventor: Gaurav CHOPRA, West Lafayette, IN (US)

(73) Assignee: Purdue Research Foundation, West Lafayette, IN (US)

(21) Appl. No.: 17/048,340

(22) PCT Filed: Apr. 17, 2019
(86) PCT No.: PCT/US19/27808

§ 371 Date: Oct. 16, 2020

Related U.S. Application Data
(60) Provisional application No. 62/658,638, filed on Apr. 17, 2018.

(51) Int. Cl. G01N 33/58; G01N 33/50; A61K 49/00

(52) U.S. Cl. CPC G01N 33/582; G01N 33/5005; A61K 49/0041; A61K 49/0054; A61K 49/0056

(57) ABSTRACT

The present disclosure generally relates to a method for modifying surface of a live cell. In particular, this method is selective for modification of a live cell surface using a multi-functional cargo agent. This method may find applications in imaging, medical diagnosis, manufacture of biotherapeutics, as well as a therapeutic agent.

### CROSS REFERENCE TO RELATED APPLICATIONS

This present patent application relates to and claims the priority benefit of U.S. Provisional Application Ser. No. 62/658,638, filed Apr. 17, 2018, the content of which is hereby incorporated herein by reference in its entirety.

### TECHNICAL FIELD

The present application relates generally to a method for modifying surface of a live cell. In particular, this invention discloses a method providing a selective and stable modification of a live cell surface using a multi-functional cargo agent. This method may find applications in imaging, medical diagnosis, manufacture of biotherapeutics, as well as a therapeutic agent.

### BACKGROUND

This section introduces aspects that may help facilitate a better understanding of the disclosure. Accordingly, these statements are to be read in this light and are not to be understood as admissions about what is or is not prior art.

Surface modification of live cells has many biological applications including imaging, control of cell surface interactions, tracking and sensing biological environments in vitro and in vivo. Over the years, several methods such as chemo-selective conjugation, PEGylation etc. have been extensively explored to modify cell surfaces with various cargos/therapeutic agents via non-covalent interactions (between positively-charged polyelectrolytes and negatively-charged cell surfaces) and covalent interactions (bonding between cargos and the functional groups on cell surfaces). However, problems remain for both non-covalent interaction based conjugation of cell surface modification, as well as, covalent conjugation of surfaces based on unnatural chemical reactions. Conjugates of cell surfaces via non covalent interactions show limited time stability. On the other hand, covalent conjugation via unnatural chemical reactions provides longer stability, but these reactions are not cyto-compatible with mammalian cells due to toxicity from metal catalyzed chemical modification of membrane proteins. Recently, cell membranes were conjugated with well-engineered therapeutic loaded nanoparticles but they cannot be used in vivo due to toxicity resulting from entrapment in the reticuloendothelial system of the liver and spleen. Therefore there are unmet needs for a selective and stable modification of a live cell surface.

### SUMMARY

The present disclosure generally relates to a method for modifying surface of a live cell. In particular, this method is selective for modification of a live cell surface using a multi-functional cargo agent. This method may find applications in imaging, medical diagnosis, manufacture of biotherapeutics, as well as a therapeutic treatment.

Surface modification of live cells has important biomedical and therapeutic applications, such as, live cell imaging and cell therapy respectively. Several methods such as chemo-selective conjugation, PEGylation etc. have been explored in recent years to modify cell surfaces with various cargos/therapeutics via non-covalent and covalent interactions. Conjugates of cell surfaces via non-covalent interactions show limited time stability. On the other hand, covalent conjugation via unnatural chemical reactions provides longer stability, but these reactions are not cytocompatible due to toxicity from unnatural and/or metal catalysed chemical modification of membrane proteins. Herein, we have designed a dual conjugation cargo molecule with a cationic side chain which forms non-covalent bonds with the negatively-charged cell surface and a phosphoric acid containing ligand which facilitates phosphor-ester covalent bonding with the cell membrane phosphate functionality. In fact, conjugation of our well-designed cargo with Jurkat T-cells shows cytocompatibility and stability of the surface conjugation over six days. We believe our dual-conjugation approach will provide a technology for live cell membrane imaging and manufacturing of therapeutic cells

These and other features, aspects and advantages of the present invention will become better understood with reference to the following figures, descriptions and claims.

### BRIEF DESCRIPTION OF THE DRAWINGS

The above and other objects, features, and advantages of the present invention will become more apparent when taken in conjunction with the following description and drawings, wherein:

FIGS. 1A-1C show design strategies for various cargos for live cell surface chemical conjugation. FIG. 1A depicts design of the non-covalent and covalent cargo backbone with both cationic side chain and phosphoric acid functionality. FIG. 1B depicts structures of the non-covalent and covalent cargo molecule. FIG. 1C depicts structures of the phospholipid linked non-covalent and covalent cargo.

FIGS. 2A-2C show chemical structures for live cell conjugation. FIG. 2A depicts cell surface functionality with negatively charged phosphate. Covalent and non-covalent conjugation of the fluorophore linked cargos on the cell surface. FIG. 2B depicts structures of the fluorophore fluorescein, fluorescein linked non-covalent and covalent cargos. FIG. 2C shows confocal images of the Jurkat T cells after treated with these cargos and followed by stained with Hoechst 33342. Both blue fluorescence of Hoechst 33342 and green fluorescence of fluorescein were observed in the nucleus of the Jurkat cells for the treatment of fluorescein whereas green fluorescence of fluoroscein was observed at the periphery of the Jurkat T cells for both the cargo treatments, indicating cell surface conjugation. Scale 5 μm.

FIGS. 3A-3F depict the stability and viability and of the surface modified Jurkat T cells. FIG. 3A shows Confocal laser microscopic images of the surface modified Jurkat T cells conjugated with both the cargos after 1, 3 and 6 days. FIG. 3A shows quantification of surface fluorescence intensity of cells in FIG. 3A. FIG. 3C shows stability of the surface conjugated cells in presence of no fluorophore tag respective cargos. FIG. 3D shows quantification of surface fluorescence intensity of cells in FIG. 3C. FIG. 3E shows viability of Jurkat T cells after their surface conjugation with the cargos. FIG. 3F shows 3D view of surface conjugated Jurkat T cells with covalent cargo. Conjugation of the covalent cargo with Jurkat T cell is not only cytocompatible, but also stable under physiological conditions (37° C. and pH 7.4) over 6 days. Scale bar 5 μm.

FIGS. 4A-4F show membrane imaging application to various live cell membrane imaging: FIG. 4A shows Jurkat T cells; FIG. 4B shows human natural killer NK-92 cells; FIG. 4C shows mouse microphase RAW264.7 cells; FIG. 4D shows human microglia HMC-3 cells; FIG. 4E shows human prostate cancer LNCaP cells; and FIG. 4F shows C4-2 cells. Scale bar 25 μm.

FIGS. 5A-5C demonstrates that cell-cell interaction leads to increased proliferation of T cell: FIG. 5A shows confocal laser microscopy images showing cell-cell interaction via the cargos. FIG. 5B shows viability of Jurkat T cells in presence of the cargo for 3 days. FIG. 5C is schematic representations showing magnetic cell separation from a mixture of cargo and free cells. Scale bar 5 μm.

FIG. 6 shows results of small molecule treatment with Jurkat T cells. Small molecule fluorophore Fluorescein and its ADP conjugate internalize inside the Jurkat T cells—indicating the necessity of cargo molecule for cell surface conjugation (Scale bar 25 μm).

FIG. 7 depicts the synthesis of covalent cargo for cell surface conjugation. Step wise synthetic schemes for the phosphate (ADP) linked covalent cargo.

FIG. 8A shows synthesis of non-covalent phospholipid cargos; FIG. 8B shows synthesis of covalent phospholipid cargos.

FIG. 9A depicts the synthesis of non-covalent; FIG. 9B shows covalent fluorophore cargos.

FIG. 10 shows the results of cargo molecular weight optimization for cell-surface conjugation. Cargo with molecular weight 150 kD conjugates on the surface of T cells while cargo with molecular weight 75 kD conjugates partially on the surface and mostly goes inside the T cells—indicating necessity of high molecular weight (>150 kD) cargo for surface conjugation. Images were captured using 60× object in Cytation 5 imaging reader. (Scale bar 30 μm).

FIG. 11 shows cargo concentration optimization for cell-surface conjugation. Cargo concentration 0.01 μg/mL showed very low yield of surface conjugation while cargo concentration 0.1 μg/mL displayed suitable conjugation as well as less precipitation of excess cargo (for 1.0 μg/mL) during the conjugation and washing procedure. These data revealed cargo concentration 0.1 μg/mL was suitable for such conjugation reactions. Cells were viewed using 60× object in Cytation 5 imaging reader (Scale bar 30 μm).

FIG. 12 shows the results of surface conjugation of Jurkat T cells with 150 kD non-covalent and covalent fluorophore cargos. Cells were treated 0.1 μg/mL concentration of the cargo for 30 minutes and images were captured using 60× object in confocal laser microscope. Scale bar 25 μm.

FIG. 13 depicts surface conjugation of Jurkat T cells with BOC-protected cargo. Ammonium cationic side chain protected cargo did not show any surface conjugation of Jurkat T cells—indicating the plausible role of this group in surface bond formation (Scale bar 25 μm).

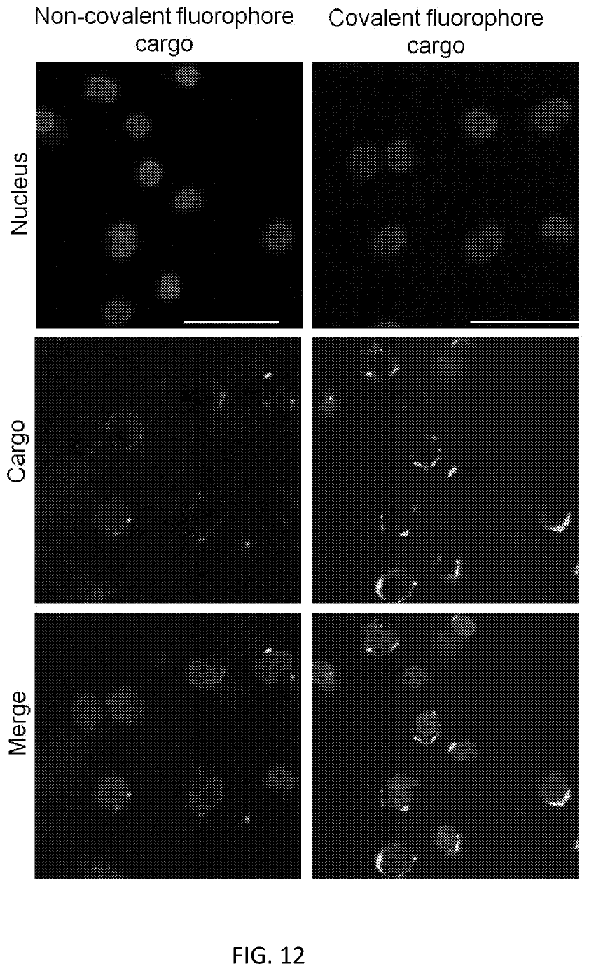

FIG. 14 shows the results of surface conjugation of activated human T cells. Live cell surface imaging of IL-2 activated Jurkat T cells. Expected surface conjugation was observed for the both non-covalent and covalent cargo treatments. (Scale bar 5 μm).

FIG. 15 demonstrates the stability of surface conjugation over days. Surface conjugation of Jurkat T cells with non-covalent and covalent fluorophore cargo. Cells were treated 0.1 μg/mL concentration of the cargo for 30 minutes and images were captured using 60× object in confocal laser microscope. Scale bar 25 μm.

FIGS. 16A and 16B depict probing additional cell surface bonding interaction between covalent and non-covalent cargos. FIG. 16A shows that the surface conjugated Jurkat T cells were treated with non-covalent cargo without fluorophore tag for. FIG. 16B shows that surface conjugated Jurkat T cells were treated with covalent cargo without fluorophore tag. Decreased green fluorescence intensity was observed for the non-covalent cargo conjugated T cells while it was less for the covalent cargo conjugated T cells (Scale bar 25 μm).

FIG. 17 depicts live cell membrane imaging applications. Membrane imaging of various live cells (Jurkat T cells, natural killer NK-92, human microglia HMC-3, mouse microphase RAW 264.7, human prostate cancer LNCaP and C4-2 cells) using the covalent cargo reagent (0.1 μg/mL) (Scale bar 25 μm).

FIG. 18 shows fixed cell membrane imaging application. Cell-surface imaging of Jurkat T cells using the covalent cargo reagent (0.1 μg/mL, Scale bar 25 μm).

FIGS. 19A and 19B demonstrate Cell-cell interactions. FIG. 19A is a schematic representation showing cell-cell interaction through cargo molecules. FIG. 19B shows bright field images of unconjugated and surface conjugated Jurkat T cells captured using 60× object in Cytation 5 imaging reader. (Scale bar 30 μm).

FIG. 20 demonstrates the process using cell-cell interaction for bio-manufacturing of therapeutic cells. Real time images of Jurkat T cells in presence of the non-covalent and covalent cargos for 6 days using IncuCyte live cell analysis system. Both the cargos were capable to produce more Jurkat T cells. Also, more confluence and clustering of Jurkat T cells were observed for the treatment of both the cargos as compared to that of the no cargo treated Jurkat T cells—suggesting these could be useful for the bio-manufacturing of such therapeutic cells.

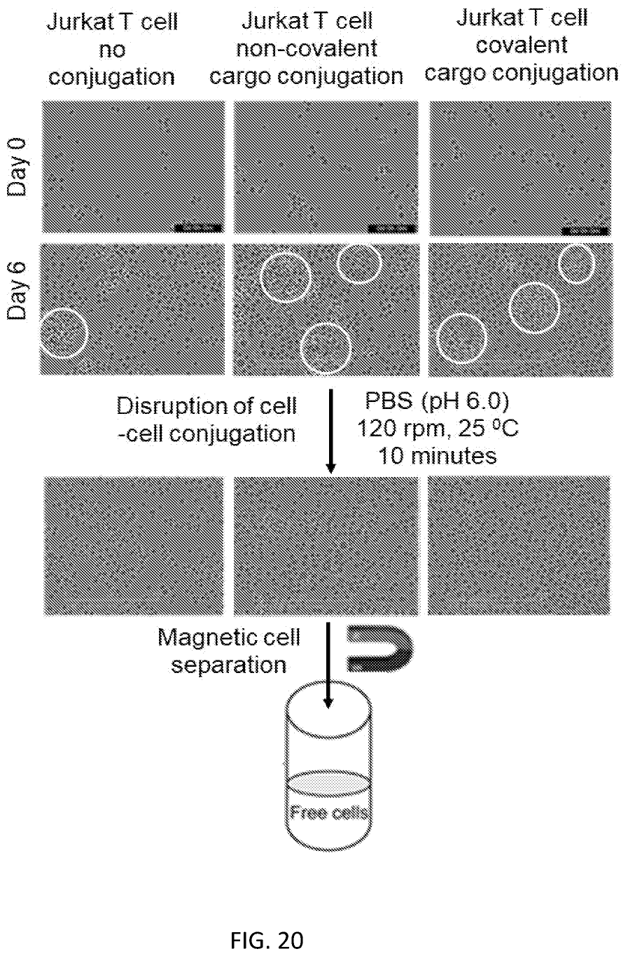

### DETAILED DESCRIPTION

For the purposes of promoting an understanding of the principles of this present disclosure, reference will now be made to the embodiments illustrated in the drawings, and specific language will be used to describe the same. It will nevertheless be understood that no limitation of the scope of this disclosure is thereby intended.

In the present disclosure the term “about” can allow for a degree of variability in a value or range, for example, within 20%, within 10%, within 5%, or within 1% of a stated value or of a stated limit of a range.

In the present disclosure the term “substantially” can allow for a degree of variability in a value or range, for example, within 80%, within 90%, within 95%, or within 99% of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In some illustrative embodiments, the present invention relates to a method for modifying surface of a live cell comprising the steps of
a. preparing a live cell to be modified;
b. preparing a cargo agent that comprises a nontoxic biodegradable polymer with one or more functional labels; and
c. adding said cargo agent to said live cell in a buffered medium having a pH value of about 6 to about 8.

In some illustrative embodiments, the present invention relates to a method for modifying surface of a live cell disclosed herein, wherein said nontoxic biodegradable polymer is a cationic polymer.

In some illustrative embodiments, the present invention relates to a method for modifying surface of a live cell disclosed herein, wherein said cationic polymer is polylysine, polyarginine or a polyamine.

In some illustrative embodiments, the present invention relates to a method for modifying surface of a live cell disclosed herein, wherein said cationic polymer has a molecular weight of about 100,000 Da˜about 200,000 Da.

In some illustrative embodiments, the present invention relates to a method for modifying surface of a live cell disclosed herein, wherein said nontoxic biodegradable polymer is an elastin-like polypeptide (ELP).

In some illustrative embodiments, the present invention relates to a method for modifying surface of a live cell disclosed herein, wherein said elastin-like polypeptide (ELP) has a molecular weight of about 100,000 Da˜about 200,000 Da.

In some illustrative embodiments, the present invention relates to a method for modifying surface of a live cell disclosed herein, wherein said functional label is a florescent sensor, voltage sensors, pH sensor, PET imaging agent, a radioactive label, or a combination thereof.

In some illustrative embodiments, the present invention relates to a method for modifying surface of a live cell disclosed herein, wherein said functional label is a fluorophore.

In some illustrative embodiments, the present invention relates to a method for modifying surface of a live cell disclosed herein, wherein said fluorophore is a rhodamine, FITC, coumarin, Cy3, Cy5, or Texas red.

In some illustrative embodiments, the present invention relates to a method for modifying surface of a live cell disclosed herein, wherein said functional label comprises a plurality of phosphate moieties.

In some illustrative embodiments, the present invention relates to a method for modifying surface of a live cell disclosed herein, wherein said phosphate moiety is adenosine di-phosphate, guanosine diphosphate, or a metal-based phosphate ligand.

In some illustrative embodiments, the present invention relates to a method for modifying surface of a live cell disclosed herein, wherein said functional label is a magnetic moiety.

In some illustrative embodiments, the present invention relates to a method for modifying surface of a live cell disclosed herein, wherein said magnetic moiety is a magnetic bead or a metal-ligand complex that enables magnetic cell separation.

In some illustrative embodiments, the present invention relates to a method for modifying surface of a live cell disclosed herein, wherein said metal-ligand complex that enables magnetic cell separation is an iron-ligand complex.

In some other illustrative embodiments, the present invention relates to a composition matter comprising a nontoxic biodegradable polymer modified with one or more functional labels.

In some other illustrative embodiments, the present invention relates to a composition matter disclosed herein, wherein said nontoxic biodegradable polymer is a cationic polymer.

In some other illustrative embodiments, the present invention relates to a composition matter disclosed herein, wherein said cationic polymer is polylysine, polyarginine or a polyamine.

In some other illustrative embodiments, the present invention relates to a composition matter disclosed herein, wherein said cationic polymer has a molecular weight of about 100,000 Da˜about 200,000 Da.

In some other illustrative embodiments, the present invention relates to a composition matter disclosed herein, wherein said nontoxic biodegradable polymer is an elastin-like polypeptide (ELP).

In some other illustrative embodiments, the present invention relates to a composition matter disclosed herein, wherein said elastin-like polypeptide (ELP) has a molecular weight of about 100,000 Da˜about 200,000 Da.

In some other illustrative embodiments, the present invention relates to a composition matter disclosed herein, wherein said functional label is a florescent sensor, voltage sensors, pH sensor, PET imaging agent, a radioactive label, or a combination thereof.

In some other illustrative embodiments, the present invention relates to a composition matter disclosed herein, wherein said functional label is a fluorophore.

In some other illustrative embodiments, the present invention relates to a composition matter disclosed herein, wherein said fluorophore is a rhodamine, FITC, coumarin, Cy3, Cy5, or Texas red.

In some other illustrative embodiments, the present invention relates to a composition matter disclosed herein, wherein said functional label comprises a plurality of phosphate moieties.

In some other illustrative embodiments, the present invention relates to a composition matter disclosed herein, wherein said phosphate moiety is adenosine di-phosphate, guanosine diphosphate, or a metal-based phosphate ligand.

In some other illustrative embodiments, the present invention relates to a composition matter disclosed herein, wherein said functional label is a magnetic moiety.

In some other illustrative embodiments, the present invention relates to a composition matter disclosed herein, wherein said magnetic moiety is a magnetic bead or a metal-ligand complex that enables magnetic cell separation.

In some other illustrative embodiments, the present invention relates to a composition matter disclosed herein, wherein said metal-ligand complex that enables magnetic cell separation is an iron-ligand complex.

In some other illustrative embodiments, the present invention relates to a kit for modifying a live cell surface comprising:

a. a cargo agent that comprises a nontoxic biodegradable polymer with one or more functional labels; and

b. a medium of pH about 6 to about 8 for conjugation of said cargo agent.

In some other illustrative embodiments, the present invention relates to a kit for modifying a live cell surface disclosed herein, wherein said nontoxic biodegradable polymer is a cationic polymer.

In some other illustrative embodiments, the present invention relates to a kit for modifying a live cell surface disclosed herein, wherein said cationic polymer has a molecular weight of about 100,000 Da˜200,000 Da.

In some other illustrative embodiments, the present invention relates to a kit for modifying a live cell surface disclosed herein, wherein said cationic polymer is polylysine, polyarginine or a polyamine.

In some other illustrative embodiments, the present invention relates to a kit for modifying a live cell surface disclosed herein, wherein said functional label comprises a florescent sensor, voltage sensors, pH sensor, PET imaging agent, a radioactive label, or a combination thereof.

In some other illustrative embodiments, the present invention relates to a kit for modifying a live cell surface disclosed herein, wherein said functional label is a fluorophore.

In some other illustrative embodiments, the present invention relates to a kit for modifying a live cell surface disclosed herein, wherein said functional label is adenosine diphosphate or guanidine diphosphate.

In some other illustrative embodiments, the present invention relates to a kit for modifying a live cell surface disclosed herein, wherein said functional label comprises adenosine diphosphate and a fluorophore.

In some other illustrative embodiments, the present invention relates to a kit for modifying a live cell surface disclosed herein, wherein said functional label comprises a magnetic moiety, adenosine diphosphate and a fluorophore.

In some other illustrative embodiments, the present invention relates to a kit for modifying a live cell surface disclosed herein, wherein said nontoxic biodegradable polymer is an elastin-like polypeptide.

In some other illustrative embodiments, the present invention relates to a kit for modifying a live cell surface disclosed herein, wherein said cationic polymer has a molecular weight of about 100,000 Da˜200,000 Da.

Surface modification of live cells has many biological applications including imaging, control of cell surface interactions, tracking and sensing biological environments in vitro and in vivo. Recently, cell surface functionalization has received significant attention from researchers and clinicians perhaps due to several biomedical applications (Li, M D, et al., Regen. Med. 2014, 9, 27; Gammon, J M, et al., Oncotarget 2016, 7, 15421). Several methods have been developed to functionalize the cell surface with cargo vehicles and therapeutic agents. Most popular conjugation methods include hydrophobic anchoring, chemo-selective conjugation, PEGylation, and others (Rabuka, D., et al., J. Am. Chem. Soc. 2008, 130, 5947; Stabler, C L, et al., Bioconjugate Chem. 2007, 18, 1713; Panza, J L, et al., Biomaterials 2000, 21, 1155). These methods are generally based on the conjugation of various functional groups with the cell surface ligands or proteins via (1) electrostatic interactions between positively-charged polyelectrolytes and negatively-charged cell surfaces such that negatively charged cell membrane bind with positively charged polycationic molecules including poly-peptides with basic amino acid side chain or cationic polyelectrolytes (Gambhir, A., et al., Biophys. J. 2004, 86, 2188); (2) hydrophobic interactions between polyelectrolyte backbones and cell membrane's lipid bilayers (Zhang, P., et al., Polymers 2017, 9, 40); and (3) covalent interactions with bond formation of cargo and functional groups on cell surfaces, for example, cargo molecule containing N-hydroxyl-succinimidyl ester (NHS) groups are widely used to form covalent bonds with the amino groups of membrane proteins (D'Souza, S. et al., Biomaterials 2014, 35, 9447). Chemical ligations on cell surfaces have been also achieved by using various unnatural functional groups (Prescher, J. A. et al., Nat. Chem. Biol. 2005, 1, 13). Recently, there have been major advances to modify live cell surface with synthetic functional polymers, macromolecular crowding, and conjugation of well-engineered therapeutic loaded nanoparticles on the cell membrane for cell therapy applications (Custodio C A et al., ChemNanoMat 2016, 2, 376; Niu, J. et al., Nat. Chem. 2017, 9, 537; Chapanian, R. et al., Nat. Commun. 2014, 5, 4683; Brannon-Peppas, L. et al., Adv. Drug Delivery Rev. 2012, 6, 206; Stephan, M T et al., Nature Medicine 2010, 16, 1035).

However, live cell surface modification achieved by both electrostatic and hydrophobic interactions show limited time stability affecting their desired function. For example, the overall stability for PEG-lipids conjugation on cell surface was no longer than 1-2 days before all the PEG molecules dissociated from the cell surface (Inui, O. et al., ACS Appl. Mater. Interfaces 2010, 2, 1514). Also, incorporating hydrophobic chain in the polymer scaffold results in uptake by the cells affecting normal cell function. On the other hand, covalent conjugation via amide bond formation with transmembrane proteins provide longer stability of the cell-surface conjugation. However, such conjugation strategies result in toxic effects during the chemical modification due to non-specific binding of the cargo molecules with other amine group containing cell membrane proteins that lead to disruption of normal cellular function (Link, A J et al., J. Am. Chem. Soc. 2003, 125, 11164). There are many limitations in the development of nanoparticle-based cell-conjugates for in vivo cell therapy. For example, nanoparticles cause toxic side effects after entrapment in the reticuloendothelial system of the liver and spleen (Riehemann, K. et al., Angew. Chem. Int. Ed. 2009, 48, 872). Finally, biocompatibility of synthetic nanoparticles that are made from inorganic materials is a major concern for their translational applications (Sanhai, W R, et al., Nat. Nanotechnol. 2008, 3, 242).

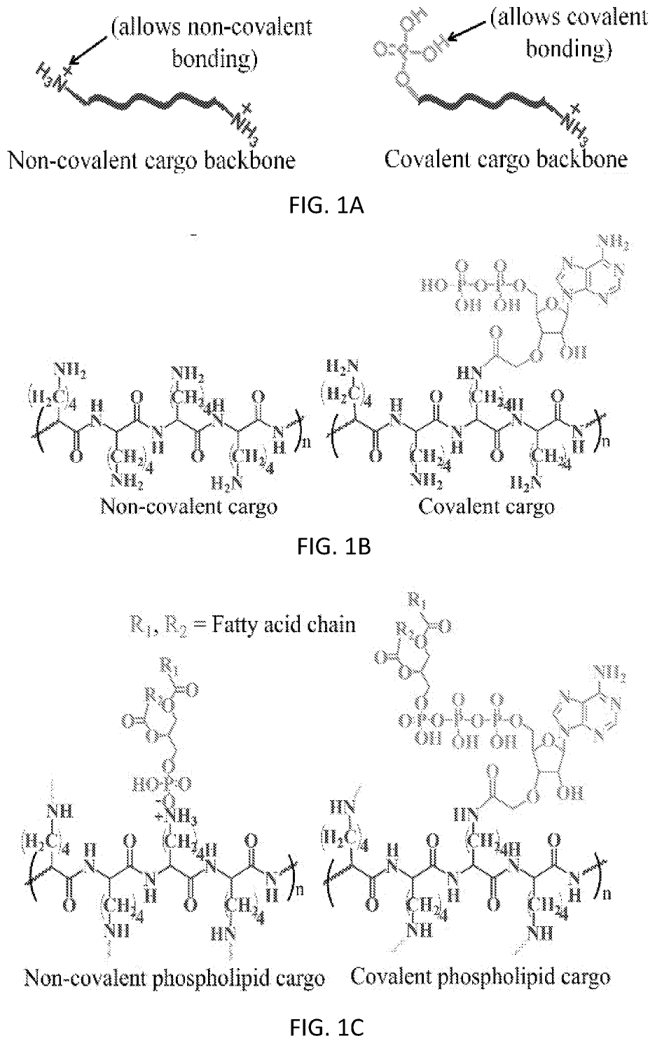

Thus, several challenges remain to develop new strategies for more stable conjugation of therapeutics/cargo for live cell surface resulting in better viability and function of these modified cells. Keeping both the stability and viability objectives in mind, we have designed a cargo molecule which will be able to bind to cell membrane directly using both covalent and non-covalent bonding. We have decorated one side of the cargo molecule with a cationic functionality which will form non-covalent interaction with the negatively charged cell surface and the other side of the cargo molecule with a phosphoric acid containing ligand such as adenosine di-phosphate (ADP) which will facilitate phosphor-ester covalent bonds under physiological condition with the cell surface phosphoric acid functionality (FIG. 1a-b). Such dual chemical conjugation approach will provide longer stability of the cell-surface conjugation as well as less toxicity because of the natural phosphor-ester bond formation instead of unnatural chemical reaction (D'Souza, S. et al., Biomaterials 2014, 35, 9447; Prescher, J. A. et al., Nat. Chem. Biol. 2005, 1, 13).

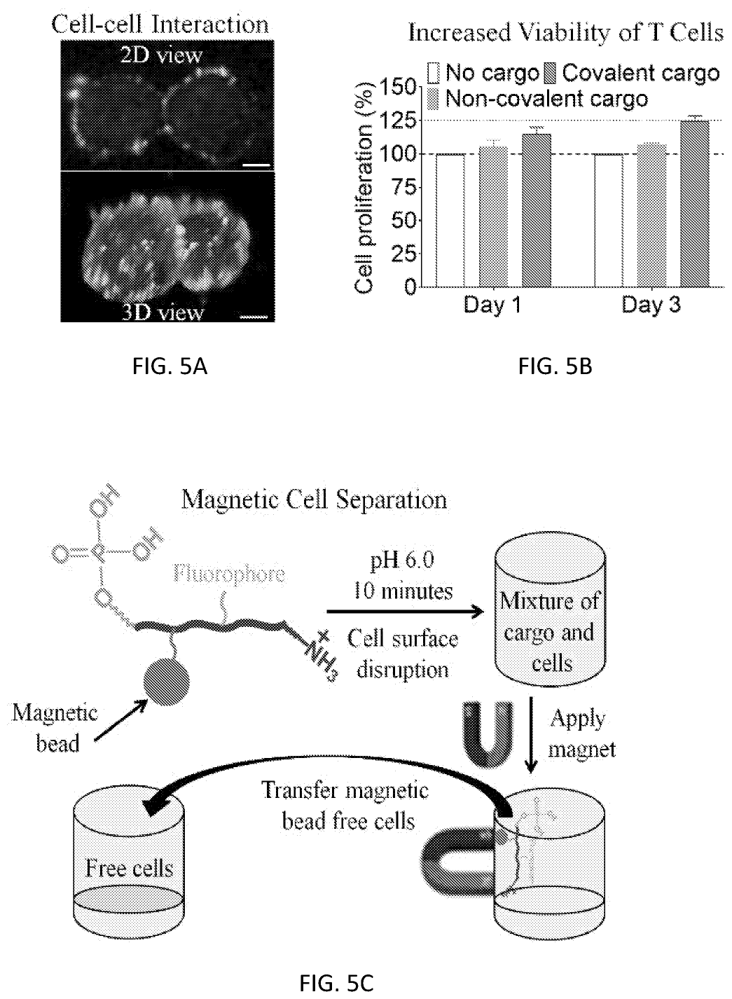

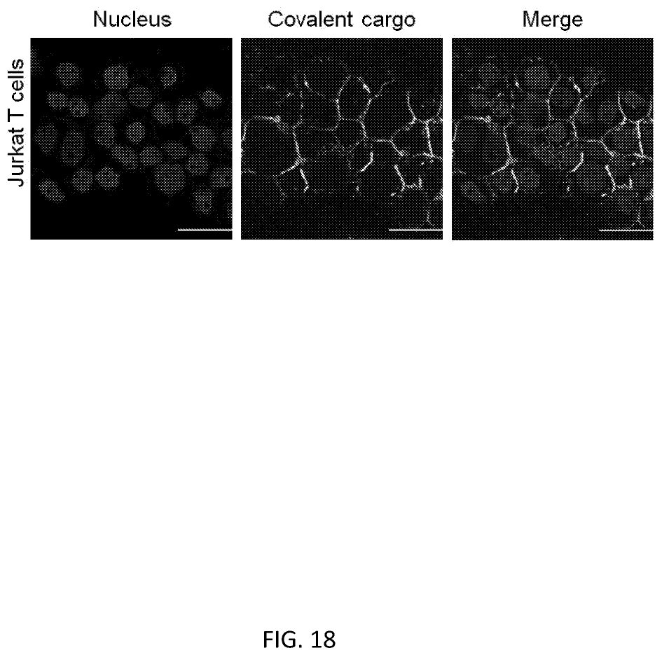

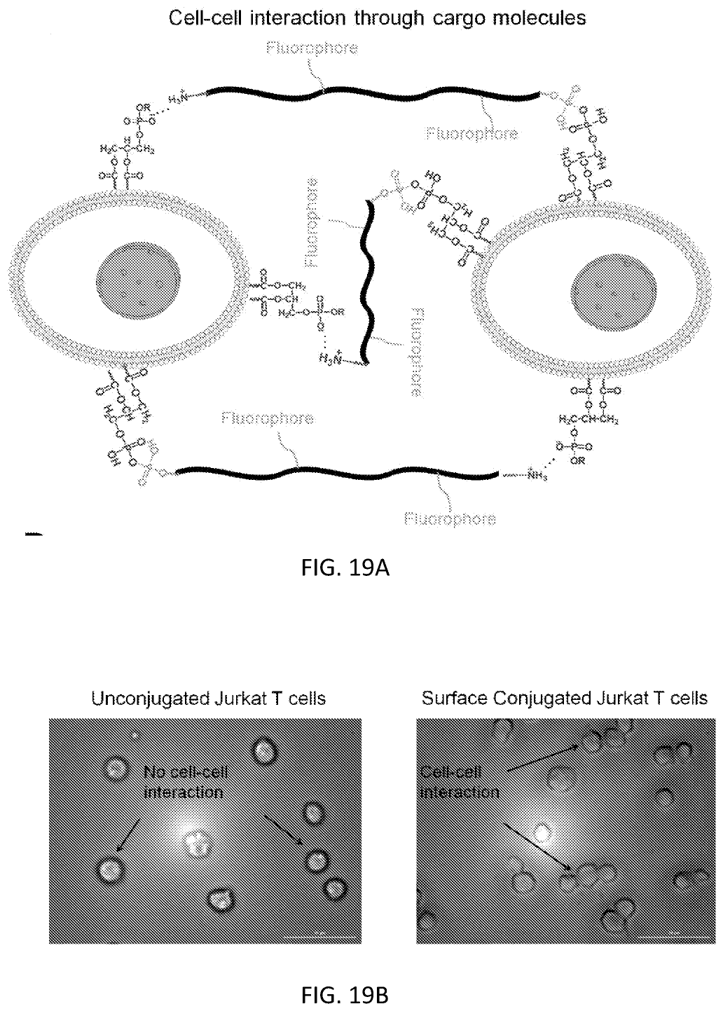

As the cell membrane is negatively charged because of the anionic phospholipid phosphatidylserine, we hypothesized that under acidic condition (pH=6.5) the phosphate anions (abundant on cell membranes) will be converted to phosphoric acid group that will be linked with the other phosphoric acid group on ADP via phosphor-ester condensation reaction. To test our hypothesis, we first synthesized an ADP-fluorescein conjugate and used it to treat Jurkat T cells Similar to fluorescein, ADP-fluorescein conjugate was internalized by T cells (FIG. 6). This result led to the design of a cargo molecule similar to a polymer/macromolecule carrier. Our polymer-based designed cargo molecule consists of one side chain with cationic group (such as NH₄⁺) and other side chain decorated with ADP. To probe the bond formation between the cargo and surface phosphates, we first carried a condensation reaction between phospholipid and our designed cargo having both side chain amine and phosphate groups in aqueous medium (FIG. 1B) and characterized by H-NMR spectroscopy (FIG. 18). In parent phospholipid fatty acid, P═O stretching appeared at ˜1170 cm⁻¹ while it was observed at ˜1156 cm⁻¹ in its cargo adduct FT-IR spectra (FIGS. 19A-19B). The decreased in P═O stretching band frequency suggested the involvement of the free phosphoric acid groups in phosphor-ammonium and/or phosphor-ester bond (where the P═O bond is present as anion and/or conjugated form) formation in the cargo adduct. Based on our hypothesis, when this cargo molecule approaches cell membrane, the non-covalent interactions of the cationic group will bring the cargo molecule close to the cell surface. Next, the vicinity of free primary alcohol group of the ADP will interact with the phosphoric acid group of cell membrane forming the phosphor-ester bond under physiological conditions resulting in both non-covalent and covalent conjugation between the cell membrane and the designed polymer cargo as outlined in FIGS. 1A-1C.

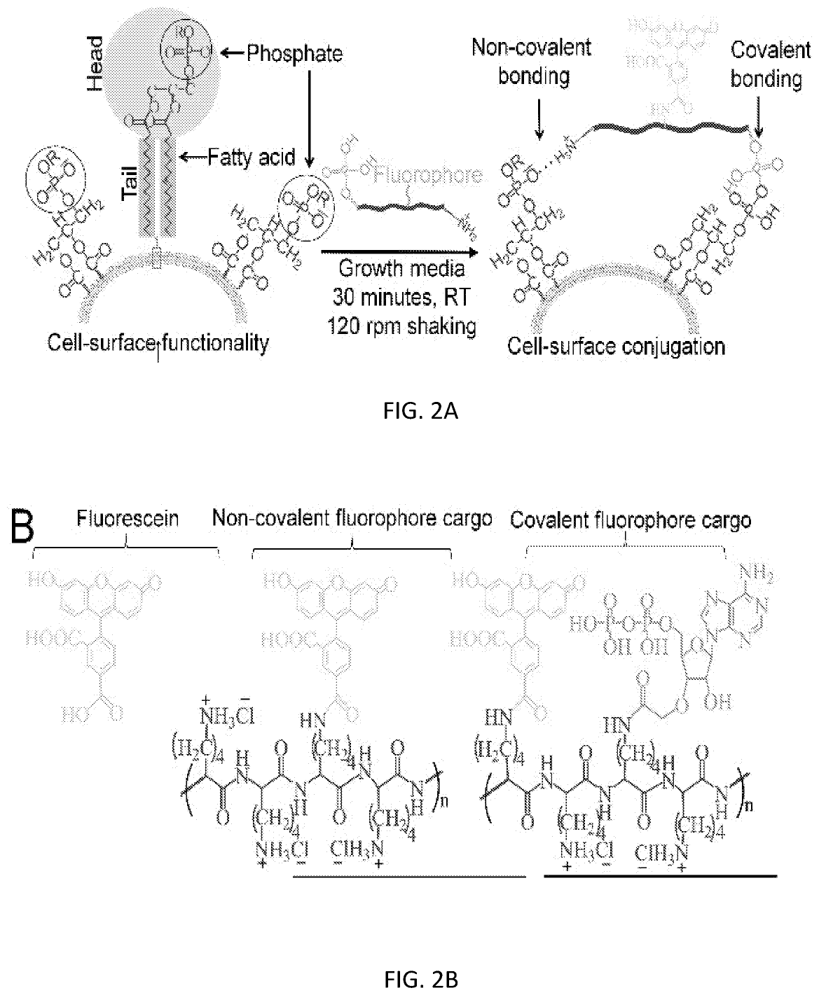

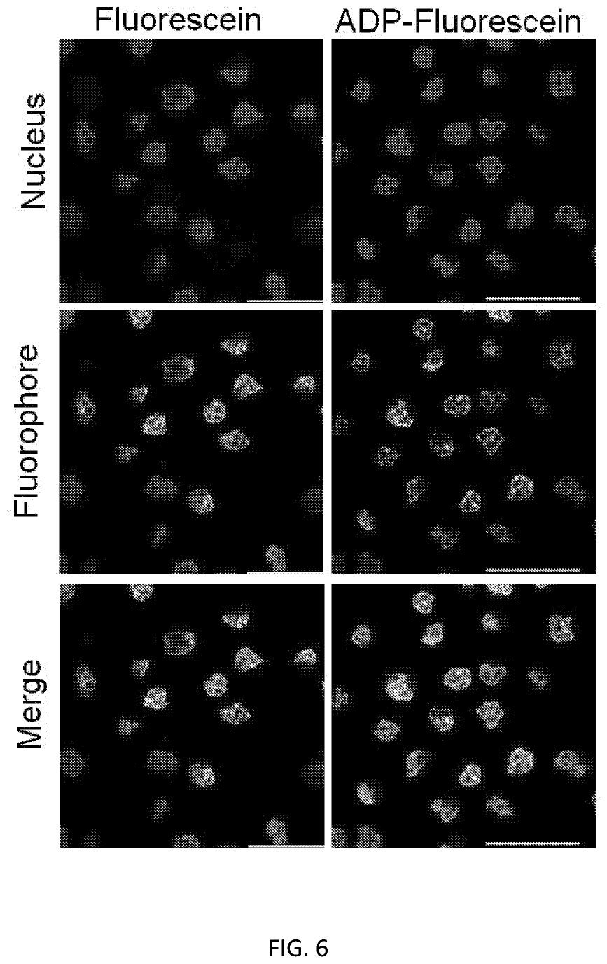

To verify our dual conjugation hypothesis, we designed and synthesized fluorophore linked to two types of cargo molecules, (1) single conjugation non-covalent cargo with only the cationic side chain, and (2) dual conjugation covalent cargo with both cationic chain (non-covalent side) and ADP (covalent side). The structures of our designed cargos are shown in FIG. 2A. The non-covalent polymer (150 kD) reacted directly with NHS-fluorescein in presence of triethylamine resulting in the non-covalent florescent cargo. For the synthesis of covalent florescent cargo, we carried out the reaction in the presence of benzotriazole activated iodoacetic acid with the cationic side chain containing polymer (150 kD) to get product I (FIG. 2B), which was further reacted with ADP in presence of base DMAP to get the product II. The ADP linked polymer chain (product II) reacted with the NHS-fluorescein in presence of triethylamine to synthesize the covalent florescent cargo. Detail synthetic steps of both this cargo molecules are outlined in FIG. 7.

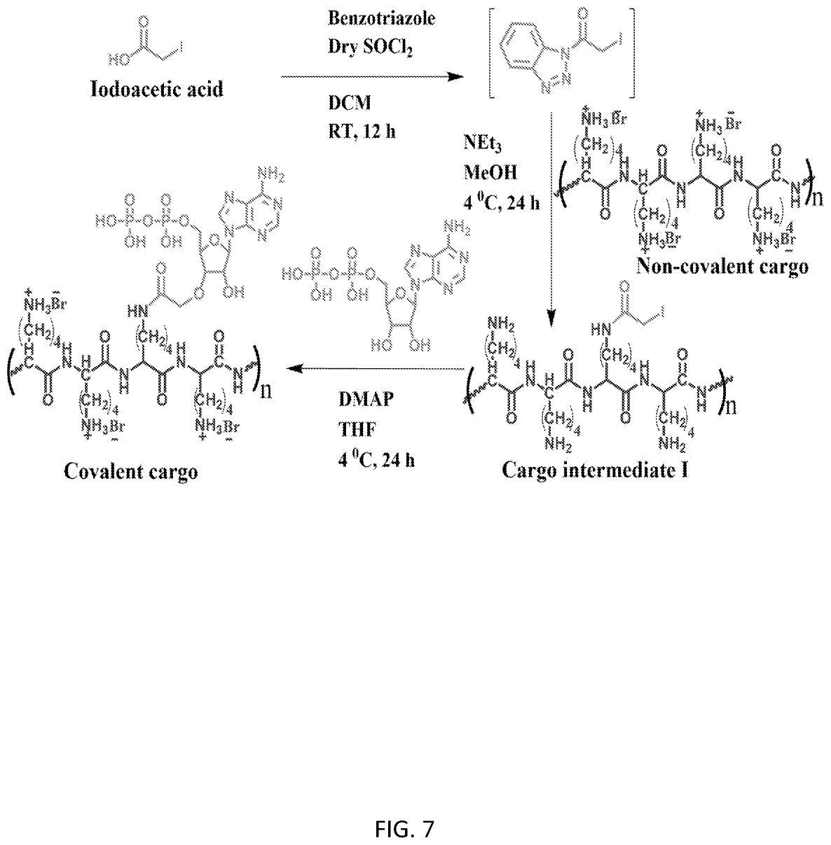

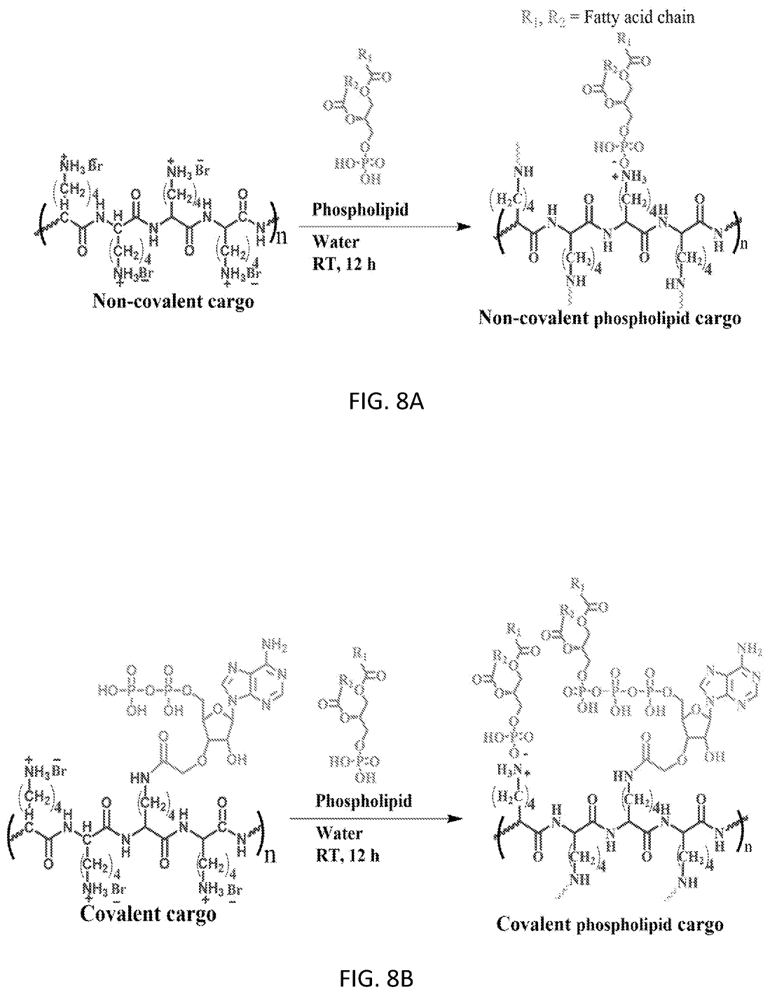

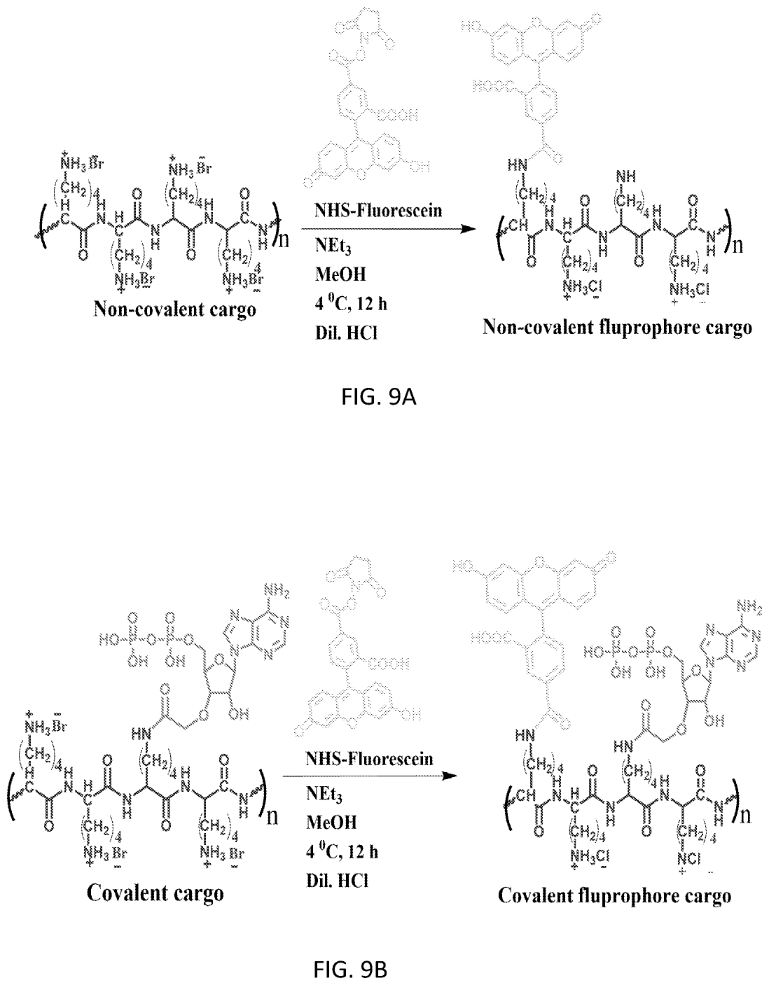

Next, we were interested to see whether the two cargo molecules conjugate on the membrane of the Jurkat T cells. Briefly, we seeded a density of 1×10⁶ cells/mL of Jurkat T cells in 12 well plates. We identified optimal conditions for the cargo (150 kD, FIGS. 8A-8B) and used it with an optimal concentration of 0.1 μg/mL to treat the cells at RT for 30 minutes on an orbital shaker (FIGS. 9A-9B). Next, cells were stained with Hoechst 33342 and washed with PBS by centrifugation and. Fluorescence confocal microscopy images of the Jurkat T cells are shown in FIG. 2C and FIGS. 8A-8B and 9A-9B. Fluorescein is internalized by the cells as we see an overlap of blue fluorescence of Hoechst 33342 and green fluorescence of fluorescein in the nucleus of the Jurkat cells. When we used the fluorescein conjugated cargo, the blue fluorescence of Hoechst was observed in the nucleus and green fluorescence of fluorescein was observed at the periphery of the Jurkat T cells. The live cell surface conjugation efficiency of the covalent cargo was better than the non-covalent cargo (where some internalization is observed, FIG. 2B), suggesting that our hypothesis to develop a dual conjugated cargo was correct to avoid cell surface internalization. Further, to test our hypothesis for dual cell conjugation, we synthesized a cargo by protecting its cationic ammonium side chains with BOC-groups and tested for cell conjugation. We observed no surface conjugation of the Jurkat T cells with the BOC-cargo clearly indicating the plausible role of the ammonium groups for cell-surface bond formations (FIG. 10).

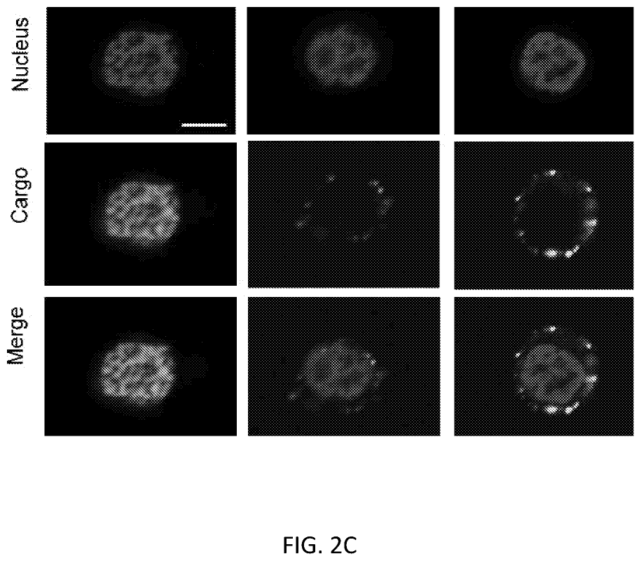

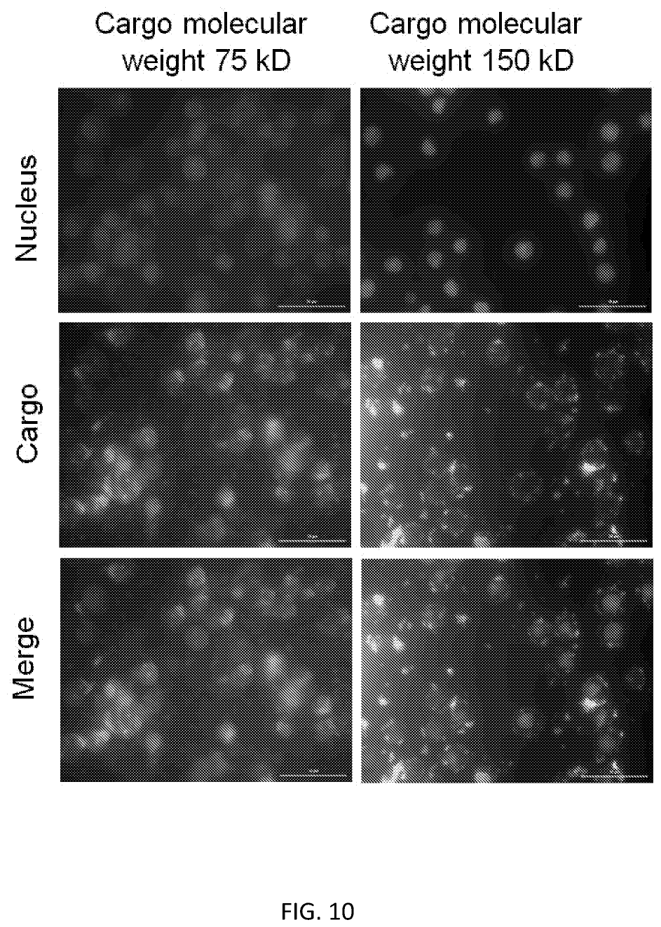

A major objective of this work is to develop cell-surface conjugation method with long time stability under physiological conditions (37° C. and pH 7.4) for several days. To our knowledge, the current stability of live cell conjugation is less than 72 hours (Inui, 0, et al., ACS Appl. Mater. Interfaces 2010, 2, 1514). We incubated surface modified Jurkat T cells at 37° C. and pH 7.4 for the period of 6 days and images were recorded after 1, 3 and 6 days. These images are shown in FIG. 3A. Both the non-covalent and dual fluorescein tagged cargo molecules show stability to the surface of the Jurkat cells—as revealed by green fluorescence on the cell membrane after 1 day of conjugation but the dual covalent cargo molecule shows stability with more than 50% florescent remaining after six days in culture (FIG. 3B). It is well known that activated T-cells results in lower surface redox levels with reduced number of membrane thiol (—SH) groups (Gelderman, K A, et al., Proc. Natl. Acad. Sci. USA 2006, 103, 12831). We verified stability of our cargo with IL-2 activated Jurkat T cells (FIG. 11) suggesting possible use for a future application of conjugated cell therapy for activated immune cells. This data clearly suggests a long time stability of the surface conjugation for the dual covalent and non-covalent conjugation compared to only non-covalent cell conjugation.

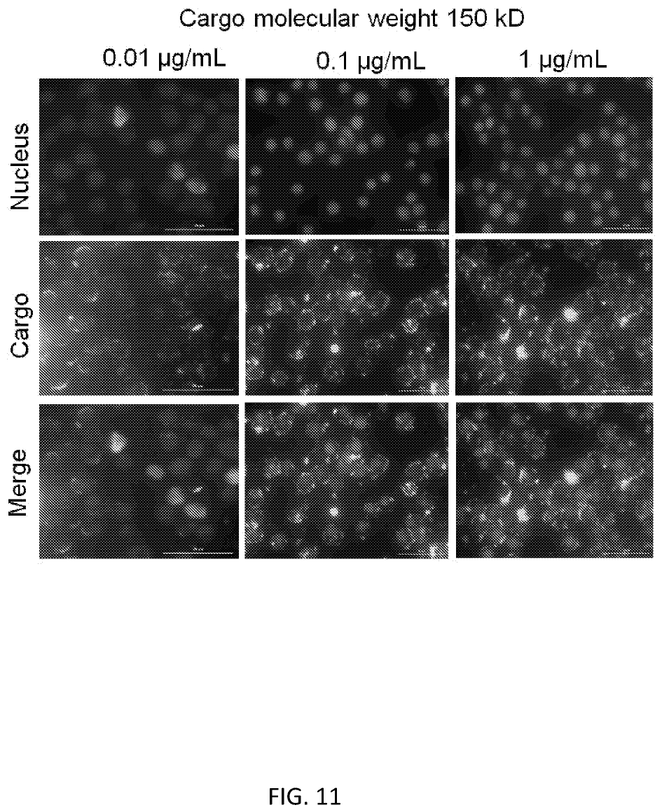

In order to investigate whether the phosphate bearing groups of the covalent cargo have additional bonding interaction with the cell surface functionality, we conjugated Jurkat T cells with fluorophore tag covalent cargo for 30 minutes and then washed with PBS. Next, those cells were treated with no fluorophore tag covalent cargo (III, FIG. S2B) for 30 minutes in growth media at 120 rpm at RT. Same experiment was performed with Jurkat T cells first treated with fluorophore tag non-covalent cargo and then no fluorophore tag non-covalent cargo (150 kD D-lysine chain polymer) for 30 minutes. At the end, cells were washed with PBS and confocal images were recorded. Results revealed that the green fluorescence intensity on the surface of Jurkat T cells conjugated with fluorophore tag non-covalent cargo reduced significantly as compared to that of the fluorophore tag covalent cargo conjugation (FIGS. 3C-3D, and FIG. 12). These data not only clearly indicated additional bonding interaction of the covalent cargo than that of the non-covalent cargo with the cell surface functionalities, but also probed our cargo design strategies/hypothesis.

Keeping cells alive after cell surface conjugation is a requirement for its further application. To see the cytotoxic effect of surface conjugation, we performed viability test of the surface modified Jurkat T cells for 6 days. We seeded 1×10⁵ surface modified Jurkat T cells in each well of 96 well plate and incubated for 1, 3 and 6 days. The cell viability was performed by the cell titer blue viability assay reagent. The cell viability results revealed that more than 95% surface modified Jurkat T cells were viable even after 6 days (FIG. 3F).

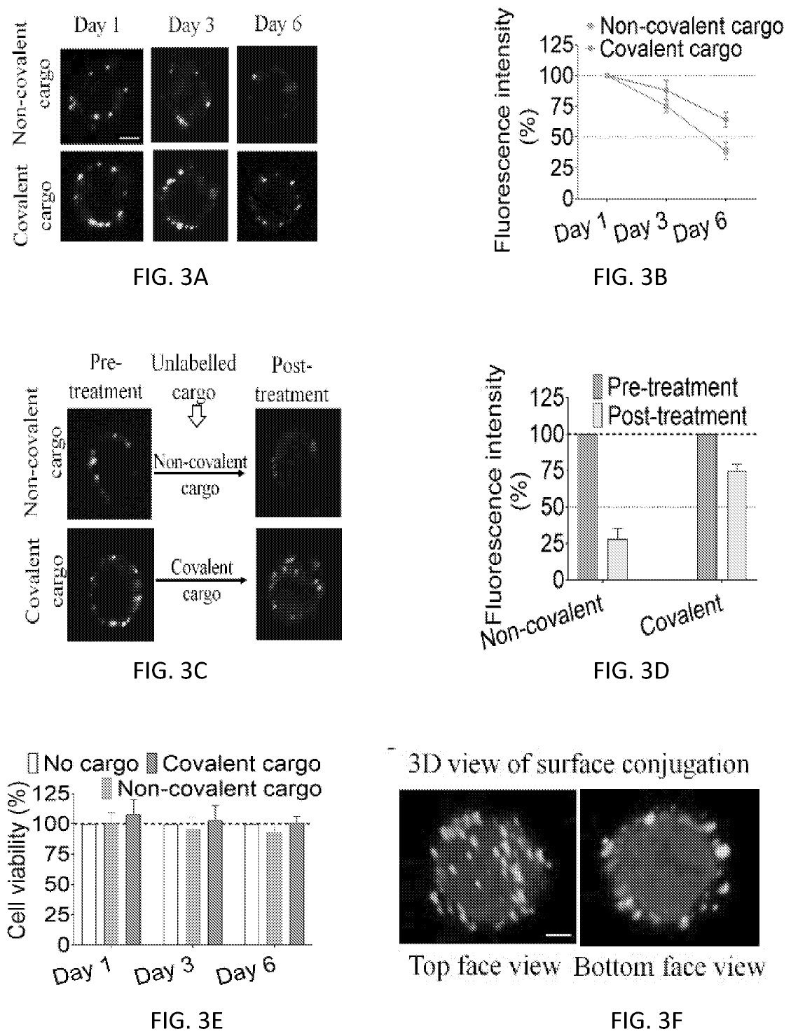

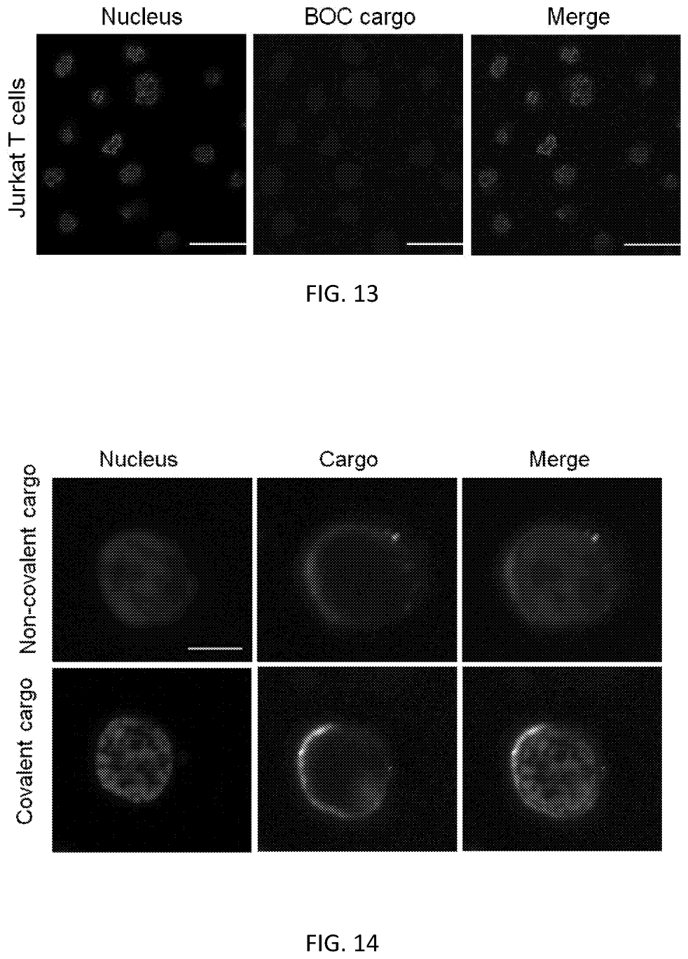

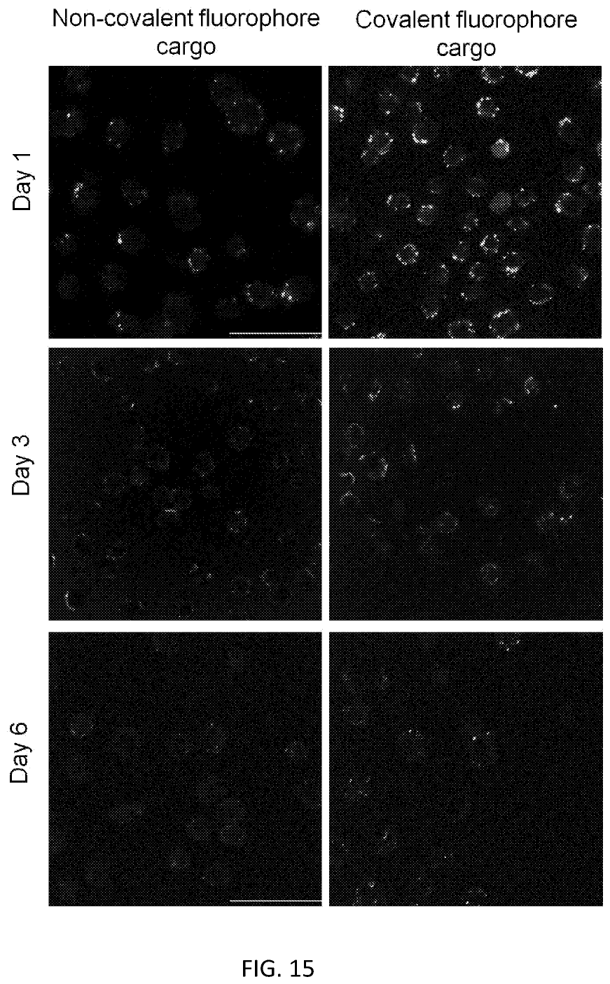

Based on the stability and non-internalization of the conjugated cargo on cell membranes, we were interested to validate the use of our florescent labelled covalent cargo as a reagent for membrane imaging of live cells. We treated various live cells (Jurkat T cells, human natural killer NK-92 cells, mouse macrophage RAW264.7 cells, human microglia HMC-3 cells, human prostate cancers LNCaP and C4-2 cells) with the covalent cargo reagent for 30 minutes and observed membrane imaging of these live cells (FIGS. 4A-4F and FIG. 14). This is a useful application of our method since there are very limited and very expensive non-toxic staining reagent for live cell membrane imaging. Our data clearly indicates that our dual cargo molecule is a suitable non-toxic reagent for any live cell membrane imaging applications. To further validate the usability of our reagent for membrane imaging of fixed cell we treated our dual covalent cargo with fixed Jurkat T cells and observed enhanced membrane imaging of cells (FIG. 15) that is comparable to other fixed cells reagents. These results suggest that our stable and non-toxic conjugate reagent can be used to image both fixed and live cells.

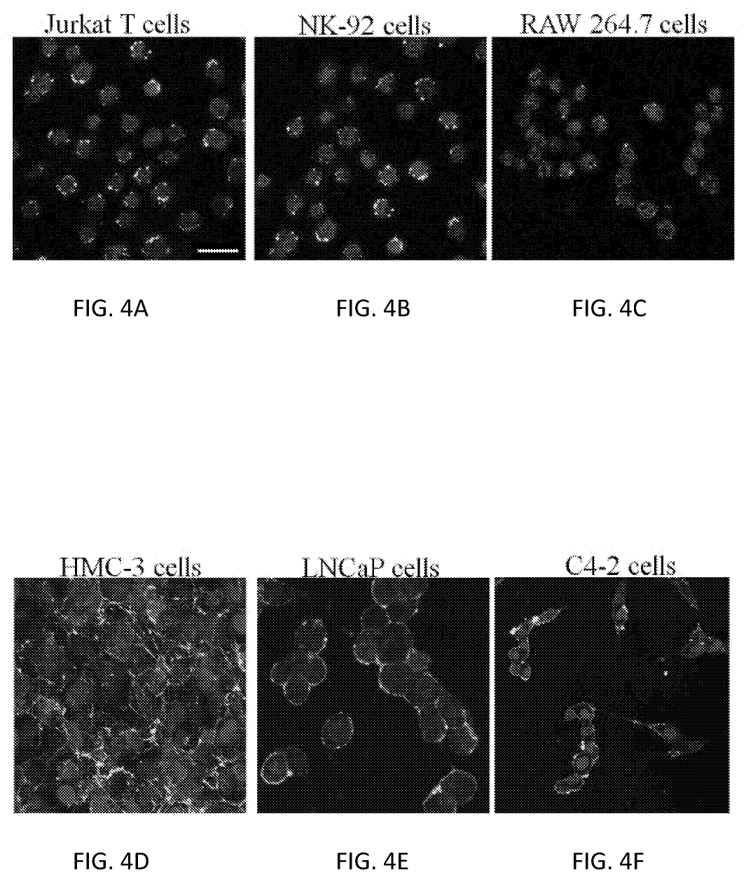

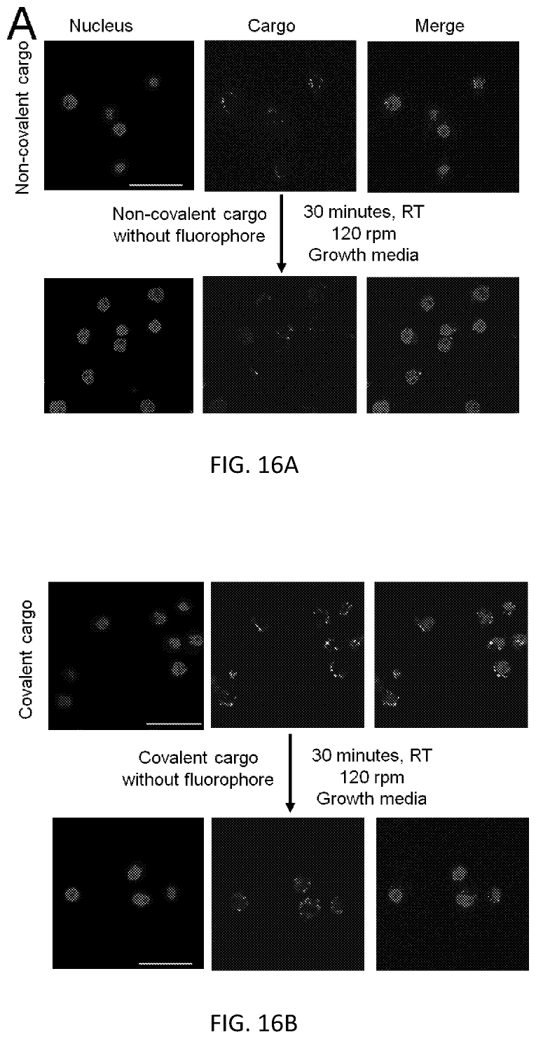

The design of our cargo molecules contain multiple bonding sites where one side of the molecule can conjugate to surface of one cell and another side of that same cargo can conjugate surface of other cells. Thus we hypothesized enhancement in cell-cell interactions leading to increase in proliferation of cells (FIG. 16A). Therapeutic cells, such as, tumor infiltrated lymphocytes (Tran, K W et al., J. Immunother. 2008, 31, 742), and chimeric antigen receptor (CAR) T-cells (Kalos, M. et al., Immunity 2013, 39, 49), have been used as immunotherapeutic “drugs.” Bio-manufacturing of these cells is a labour-intensive process and any enhancement to reduce the ex vivo expansion time is desirable for a large-scale cost-effective production of therapeutic cells. To see whether the dual covalent cargo is able to facilitate cell-cell interaction, we treated Jurkat T cells with it for 30 minutes and then captured bright field images of both the un-treated and treated Jurkat cells. This data revealed that untreated Jurkat cells as isolated cells whereas covalent cargo treated Jurkat cells as clustered cells enhancing cell-cell interactions (FIG. 16B). Fluorescence images of the Jurkat cells also revealed cell-cell clustering phenomena for the covalent cargo treatment (FIG. 5B).

To verify our cell-cell interaction-based proliferation hypothesis we treated 0.1 μg/mL of both the non-covalent and dual conjugation cargo molecules with T cells and performed a 3 days proliferation assay. We compared the results with cell culture growth media as vehicle control and measured the fluorescent signal using ELISA reader. The percentage of live cells in a cargo-treated sample was calculated by considering the fluorescence intensity of the vehicle treated cells as 100% on respective days. We observed a significant increase of 10% and 25% increased proliferation of the Jurkat T cells for non-covalent and covalent cargo treatments respectively (FIG. 5C). To provide the utility of our dual conjugation strategy for enhancing cell therapy process, we functionalized magnetic beads to the cargo. Our dual conjugation strategy is reversible, in that, we can disrupt the cell-cell interactions by treating the culture with PBS of pH 6.0 and re-conjugate the cells by pH stimulation. The magnetic cargo molecule makes it easier to separate cells from the cargo molecules resulting in culture with proliferated cells without the cargo (FIG. 5A). This process can be repeated several times for faster proliferation of therapeutic cells. Thus, our covalent magnetic cargo molecule can be easily used to enhance the efficiency of bio-manufacturing for therapeutic ells.

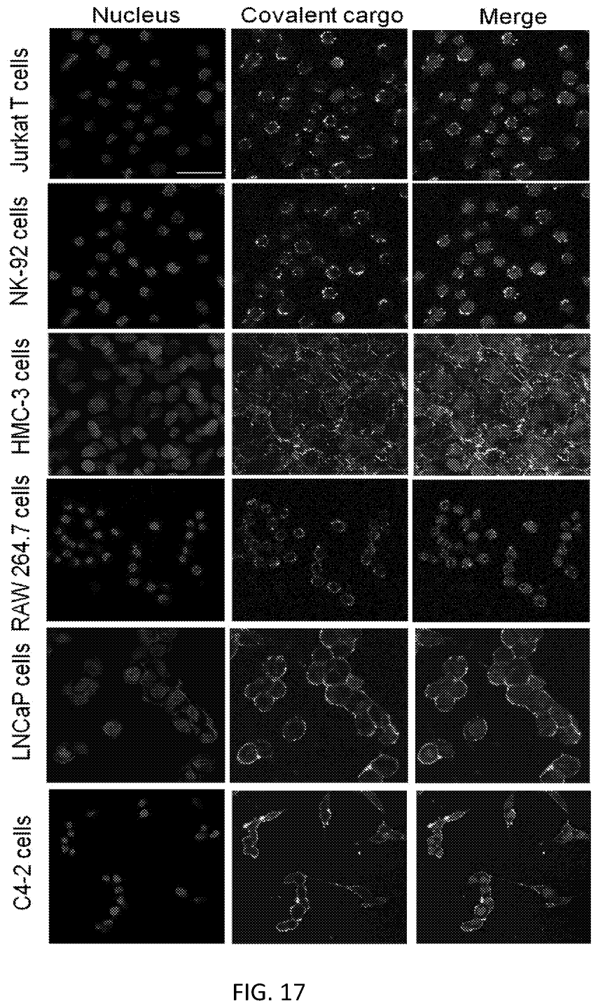

Finally, we were interested to study the change in proliferation and clustering of T cells with time. We recorded the real-time videos of conjugated and unconjugated T-cells by IncuCyte live cell analysis system suggesting increase in confluency and clustering of dual conjugated treated T-cells compared to no cargo control (FIG. 17). Thus, our data clearly suggest the dual conjugation cargo reagent can be used to enhance cell-cell interaction leading to proliferation of therapeutic cells for bio-manufacturing

To conclude, we have developed a cytocompatibile and stable method of chemical conjugation with live cell membranes. One side chain of our cargo molecule is a positively charged ammonium moiety, which provide electrostatic binding resulting in non-covalent conjugation with the cell surface; while another side of it contained a phosphate group bearing ADP moiety which was utilized to form phosphor-ester covalent conjugation with the cell surface phosphates. This dual modification strategy enables long-time stability on cell membrane and non-toxicity based on cell viability. We have shown application of this method for live membrane imaging as well as enhancement of cell-cell interaction leading to cell proliferation. Our next steps involve doing in vivo imaging and stability of our cargo molecules to track CAR T-cells in vivo as well as use of our protocol to enhance manufacturing of primary cells for clinical application.

Materials and Methods

All the chemicals including NHS-fluorescein, adenosine di-phosphate (ADP), poly-d-lysine, phospholipid etc. were purchased from commercial suppliers and used without further purification. We purchased analytical grade solvents from commercial suppliers for synthesis. Fluorescence images of cells were captured using 40× and 60× objective using both Cytation 5 imaging reader and confocal laser microscope (Nikon AR1-MP) and 3D videos are generated as supporting information. Cell viability and proliferation assays was performed by using cell titer blue (CTB) reagent. Real time images and videos of viable T cells were recorded using IncuCyte S3 live cell analysis system and included as supporting information.

Cell Culture

We obtained Jurkat T cells from Dr. Majid Kazemian (Purdue University, USA) and NK-92 cells from Dr. Sandro Matosevic (Purdue University, USA). LNCaP, C4-2 and RAW 264.7 cell lines were provided by Professor Timothy Ratliff (Purdue University Center for Cancer Research, USA). HMC-3 cell line was given by Professor Jianming Li, Purdue University College of Veterinary Medicine. Cells were cultured following American Type Culture Collection (ATCC) protocol at 37° C. with 5% CO₂ atmosphere in a humidified incubator. For normal growth, Jurkat T cells were cultured in RPMI-1640 media (Gibco) supplemented with 10% FBS (Atlanta Biologics), 20 mM HEPES and 1% penicillin/streptomycin (Invitrogen). NK-92 cells were cultured in RPMI-1640 supplemented with 100 IU/mL IL-2. All the cargo compounds were dissolved in PBS at high concentration (1 mg/mL) followed by filtrations using a 0.22 μm syringe filter and dilutions from this stock solution were prepared in culture medium.

Synthesis of Covalent Cargo:

Step 1:

Thionyl chloride (1.1 mmol) was added to a solution of benzotriazole (3 mmol) in dry DCM (10 mL) at room temperature and the reaction mixture was stirred for 10 min. Then iodoacetic acid (1.0 mmol) was added and the mixture was stirred for 12 h at room temperature. The white precipitate obtained was filtered off, and the filtrate was concentrated under reduced pressure. After the evaporation of the solvent, the crude residue of benzotriazolide was isolated as intermediate and used in the next step reaction where it (0.012 mmol) was added to a solution of D-lysine chain polymer (30 mg, 150 kD) in 5 mL of a MeOH in the presence of Et₃N (100 μL). The reaction mixture was stirred at 4° C. for 12 h. After the evaporation of the solvent, crude residue of cargo intermediate I was isolated as precipitate which was purified by washing with methanol and used in the next step.

Step 2:

ADP (5.3 mg) was dissolved in 5 mL THF in a RB followed by addition of DMAP (2.4 mg) to it. This mixture was stirred for 60 minutes at 4° C. followed by addition of I (30 mg). The resulting reaction mixture was further stirred for 24 h at 4° C. The reaction mixture was acidified with ice cold dil. HCl to neutralize any remaining alkoxide of the ADP as well as form cationic ammonium chloride non-covalent probe in the cargo backbone. Finally, the solvent was evaporated to dryness to get the crude product of covalent cargo which was purified by washing with methanol and acetonitrile.

Synthesis of Non-Covalent Phospholipid Cargo:

4.0 mg of D-lysine chain polymer (150 kD) was dissolved in 5 mL water. 10 μL triethyl amine was added and mixture was stirred for 5 minutes at RT to make free all the amine groups. Next, 2.0 mg phospholipid was dissolved in 2 mL methanol and added to it and the reaction mixture was stirred for 30 minutes at RT. The reaction mixture was evaporated to get the crude residue which was washed with acetonitrile to remove any un-reacted phospholipid and get the non-covalent phospholipid cargo.

Synthesis of Covalent Phospholipid Cargo:

6.0 mg of covalent cargo was dissolved in 5 mL water followed by addition of 2.0 mg phospholipid to it and the reaction mixture was stirred for 2 h at RT. The reaction mixture was evaporated at RT to get the crude residue which was washed with cold acetonitrile to remove any un-reacted phospholipid and get the title product.

Synthesis of Non-Covalent Fluorophore Cargo:

8.0 mg HBr salt of D-lysine chain polymer (150 kD) was dissolved in 5 mL methanol by adding 100 μL triethyl amine. This mixture was stirred for 30 minutes at 4° C. followed by addition of 3.0 mg NHS-Fluorescein. The resulting reaction mixture was further stirred for 12 h at 4° C. Un-reacted triethyl amine was neutralized by dropwise addition of ice cold dil. HCl. The orange-yellow precipitate obtained from this reaction was separated and washed with cold methanol and dried at room temperature under vacuum to get the title non-covalent fluorophore cargo product.

Synthesis of Covalent Fluorophore Cargo:

6.0 mg of covalent cargo was dissolved in 5 mL methanol by adding 100 μL triethyl amine. This mixture was stirred for 30 minutes at 4° C. followed by addition of 1.0 mg NHS-Fluorescein. The resulting reaction mixture was further stirred for 12 h at 4° C. Un-reacted triethyl amine was neutralized by dropwise addition of ice cold dil. HCl. The orange-yellow precipitate obtained from this reaction was separated and washed with cold methanol and dried at room temperature under vacuum to get the title product covalent cargo.

Synthesis of Butoxy Carbonyl-Protected Non-Covalent Fluorophore Cargo (BOC-Cargo):

4.0 mg HBr salt of D-lysine chain polymer (150 kD) was dissolved in 5 mL methanol followed by adding 100 μL triethyl amine. This mixture was stirred for 30 minutes at 4° C. followed by addition of 2.0 mg NHS-fluorescein dye. The resulting reaction mixture was further stirred for 12 h at 4° C. Next, BOC-anhydride (5.0 mg) was added to it and the reaction mixture was stirred for another 2 h. The precipitate obtained from this reaction was separated and washed with cold methanol and dried at room temperature under vacuum to get the BOC-protected non-covalent fluorophore cargo.

General Synthesis of Magnetic Bead Linked Cargo:

4.0 mg of each non-covalent and covalent fluorophore cargo was dissolved in 5 mL methanol by adding 100 μL triethyl amine. The reaction mixture was stirred for 30 minutes at 4° C. to make free all the amine groups. Next, 1.0 mg of NHS-magnetic beads was added to each of the reaction mixture and stirred for 12 h at 4° C. Un-reacted triethyl amine was neutralized by dropwise addition of ice cold dil. HCl. The orange-yellow precipitate obtained from this reaction was separated and washed with cold methanol and dried at room temperature under vacuum to get the magnetic bead linked respective cargos.

General Procedure for Cell-Surface Conjugation and Live Cell Imaging

Jurkat cells (100,000/well) were taken in each well of 12 well plate and treated with 0.1 μg/ml concentration of fluorescein, non-covalent and covalent fluorophore cargos in growth media and shaked at 120 rpm using an orbital shaker for 30 minutes at RT. Next, cells were stained with 0.1 μg/ml concentration of Hoechst 33342 (for nucleus) in growth media and washed with sterile PBS and transferred in glass bottom dish. Cells were viewed under 60× oil object (optical zoom 3) in confocal laser microscope (Nikon AR1-MP).

Fixed Cell Surface Conjugation and Imaging:

Jurkat T cells were mixed with 4% fixing solution (4% paraformaldehyde made in PBS) and immediately transferred in glass bottom dish. Cells were seeded as well as fixed by centrifugation at 1000 rpm at 10° C. for 5 minutes. Next, fixed cells were gently rinsed with PBS to remove any fixation agent and treated with the covalent fluorophore cargo (0.1 μg/mL) for 30 minutes in PBS at RT. Cells were stained with DAPI and washed with PBS and again centrifuged to make sure their attachment on the glass bottom surface. Finally, confocal images were captured using 60× oil object.

Physiological Stability of the Surface Modified Jurkat T Cells

Surface conjugated Jurkat T cells (100,000 cells/well) were grown in 12 well culture plate in growth media and images were captured after 1, 3 and 6 days of incubation using confocal laser microscope. Fluorescence intensity was measured using NIS-Elemental software.

Probing Additional Bonding of Covalent Cargo: Cargo Displacement Reactions with Surface Modified Jurkat T Cells in Presence of No Fluorophore Tag Cargos

Jurkat T cells were conjugated with the non-covalent and covalent fluorophore cargos for 30 minutes. Next, these surface conjugated Jurkat T cells (100,000 cells/well) were taken in 12 well culture plate and treated with no fluorophore tag non-covalent and covalent cargo for another 30 minutes in growth media at 120 rpm shaking at RT. Cells were then and stained with Hoechst 33342 (for nucleus) and washed with PBS. Finally, cells were transferred in glass bottom dish and confocal images were recorded to monitor the retention of surface conjugation.

Viability Assay of the Surface Modified Jurkat T Cells

The cell viability experiment was performed using the cell titer blue reagent. Surface conjugated Jurkat T cells (100,000 cells/well) were seeded in each well of 96-well plates using growth media and incubated in a humidified incubator at 37° C. and 5% CO₂ atmosphere. At the end of the incubation, cell titer blue reagent was added directly to each well and the plates were incubated for additional 3 h at 37° C. to allow cells to convert resazurin to resorufin, and the fluorescent signal was measured at 590 nm after exciting at 560 nm using a multiplate ELISA reader (Bio-Tek Synergy HT plate reader, Bio-Tek, Winooski, Vt.). The percentage of live cells in a cargo-conjugated sample was calculated by considering the fluorescence intensity of the vehicle treated un-conjugated Jurkat cell sample as 100%.

Live Cell Membrane Imaging

All the adherent cells (mouse microphase RAW264.7 cells, human microglia HMC-3 cells, human prostate cancers LNCaP and C4-2 cells) were grown on glass bottom dish following ATCC protocol. All these live cells were treated with 0.1 μg/mL of the covalent fluorophore cargo for 30 minutes in their respective growth media at RT at 120 rpm shaking. Cells were then stained with and washed with PBS. Finally, images were captured using 40× oil object in confocal laser microscope. For, both suspension Jurkat T and human natural killer NK-92 cells, general live cell imaging method was followed.

T-Cell Manufacturing: T-Cell Proliferation in Presence of the Cargo Reagents

Jurkat T cells 100,000 cells/well were seeded in 96-well plates in growth media and incubated for 3 days in presence of 0.01 μg/mL concentration of these cargos in a humidified incubator at 37° C. and 5% CO₂ atmosphere. At the end of the incubation, cell titer blue reagent was added directly to each well and the plates were incubated for 3 h at 37° C. to allow cells to convert resazurin to resorufin, and the fluorescent signal was measured using a multiplate ELISA reader (Bio-Tek Synergy HT plate reader, Bio-Tek, Winooski, Vt.). The percentage of live cells in a cargo reagent treated sample was calculated by considering the vehicle treated Jurkat T cell sample as 100%.

Proliferation and Clustering of the T Cells in Presence of the Cargo Reagents (IncuCyte Live-Cell Analysis)

200,000 cells/well were taken in 48-well plates in growth media and treated with 0.01 μg/mL of the magnetic bead linked respective cargos and incubated for 6 days at 37° C. inside the IncuCyte incubator. Both the proliferation and clustering of the Jurkat T cells were monitored by real time image and video recoded by IncuCyte S3 live cell analysis system. After 6 days of treatment, the clustered Jurkat cells in each well were treated with PBS-HCl to maintain pH of 6.0 for 10 minutes at 120 rpm in an orbital shaker. Next, images of the HCl treated cells were recorded again by the IncuCyte S3 live cell analysis system. Finally, magnetic cell separation technique was employed to isolate cargo free pure Jurkat T cells.

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected. It is intended that the scope of the present methods and apparatuses be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

1. A method for modifying surface of a live cell comprising the steps of
a. preparing a live cell to be modified;
b. preparing a cargo agent that comprises a nontoxic biodegradable polymer with one or more functional labels; and
c. adding said cargo agent to said live cell in a buffered medium having a pH value of about 6 to about 8.

2. The method of claim 1, wherein said nontoxic biodegradable polymer is a cationic polymer selected from the group consisting of polylysine, polyarginine or polyamine having a molecular weight of about 100,000 Da˜about 200,000 Da.

3. (canceled)

4. (canceled)

5. The method of claim 1, wherein said nontoxic biodegradable polymer is an elastin-like polypeptide (ELP) having a molecular weight of about 100,000 Da˜about 200,000 Da.

6. (canceled)

7. The method of claim 1, wherein said functional label is a florescent sensor, a voltage sensor, a pH sensor, a PET imaging agent, a radioactive label, a magnetic label, or a combination thereof.

8. The method of claim 1, wherein said functional label is a fluorophore selected from the group consisting of a rhodamine, FITC, coumarin, Cy3, Cy5, or Texas red.

9. (canceled)

10. The method of claim 1, wherein said functional label comprises a plurality of phosphate moieties selected from the group consisting of adenosine di-phosphate, guanosine diphosphate, or a metal-based phosphate ligand.

11. (canceled)

12. The method of claim 1, wherein said functional label is a magnetic moiety comprising a magnetic bead or a metal-ligand complex that enables magnetic cell separation.

13. (canceled)

14. The method of claim 12, wherein said metal-ligand complex that enables magnetic cell separation is an iron-ligand complex.

15. A composition matter comprising a nontoxic biodegradable polymer modified with one or more functional labels, wherein said nontoxic biodegradable polymer is an elastin-like polypeptide (ELP) or a cationic polymer of polylysine, polyarginine or a polyamine.

16.-19. (canceled)

20. The composition matter of claim 15, wherein said elastin-like polypeptide (ELP) or said cationic polymer of polylysine, polyarginine or polyamine has a molecular weight of about 100,000 Da˜about 200,000 Da.

21. The composition matter of claim 15, wherein said functional label is a fluorophore, a voltage sensor, a pH sensor, a PET imaging agent, a radioactive label, a magnetic label, or a combination thereof.

22. (canceled)

23. The composition matter of claim 21, wherein said fluorophore is a rhodamine, FITC, coumarin, Cy3, Cy5, or Texas red.

24. The composition matter of claim 15, wherein said functional label comprises a plurality of phosphate moieties selected from the group consisting of adenosine di-phosphate, guanosine diphosphate, or a metal-based phosphate ligand.

25. (canceled)

26. (canceled)

27. The composition matter of claim 21, wherein said magnetic label is a magnetic bead or a metal-ligand complex that enables magnetic cell separation.

28. The composition matter of claim 27, wherein said metal-ligand complex that enables magnetic cell separation is an iron-ligand complex.

29. A kit for modifying a live cell surface comprising:
a. a cargo agent that comprises a nontoxic biodegradable polymer with one or more functional labels, wherein said nontoxic biodegradable polymer is an elastin-like polypeptide (ELP) or a cationic polymer of polylysine, polyarginine or a polyamine having a molecular weight of about 100,000 Da˜200,000 Da; and
b. a medium of pH about 6 to about 8 for conjugation of said cargo agent.

30. (canceled)

31. (canceled)

32. (canceled)

33. The kit of claim 29, wherein said functional label comprises a fluorophore, a voltage sensor, a pH sensor, a PET imaging agent, a radioactive label, a magnetic label, or a combination thereof.

34. (canceled)

35. The kit of claim 29, wherein said functional label is adenosine diphosphate.

36. The kit of claim 29, wherein said functional label comprises adenosine diphosphate and a fluorophore.

37. The kit of claim 29, wherein said functional label comprises a magnetic moiety, adenosine diphosphate and a fluorophore.

38. (canceled)

39. (canceled)
